(19) United States
(12) Patent Application Publication
(10) Pub. No.: US 2016/0128873 A1
(43) Pub. Date: May 12, 2016

(54) Bandage with Releasable Pad and Methods Therefor

(71) Applicant: Medline Industries, Inc., Mundelein, IL (US)

(72) Inventors: Tambra Martin, Trevor, WI (US); Lisa Bauer, Buffalo Grove, IL (US)

(21) Appl. No.: 14/538,636

(22) Filed: Nov. 11, 2014

(51) Int. Cl. A61F 13/00; A61K 31/14; A61K 33/00; A61K 9/70

(52) U.S. Cl. CPC A61F 13/00042; A61K 9/7084; A61K 31/14; A61K 33/00; A61F 13/00029; A61F 2013/00285

(57) ABSTRACT

A bandage (100) includes a backing layer (101). A film layer (102) defines a first major face (109) and a second major face (110). The first major face can include one or more design elements (111) and can be coupled to the backing layer. An adhesive layer (114) can be disposed along the second major face, with an absorbent layer centrally disposed along the adhesive layer on the second major face at an interface (112). The interface can release the absorbent layer when the absorbent layer absorbs a wetting agent (801) such as wound exudate. An absorbent layer (103) can include an active ingredient, such as BZK (1403), and can be color coded with one or more colorants (1402).

### BACKGROUND

1. Technical Field

This disclosure relates generally to wound care, and more particularly to bandages for wound care.

2. Background Art

Dressings are frequently used in wound treatment. Dressings are frequently applied to wounds to provide a protective covering, thereby helping the wound to heal. For example, many types of wounds are normally bandaged using a gauze and adhesive tape combination. Such adhesive bandages are frequently used to treat minor cuts, scrapes, burns, blisters, and other wounds. These bandages frequently include a polymeric base layer is coated with an adhesive. An absorbent pad is then centrally disposed along the base layer atop the adhesive.

A user places the pad atop a wound and wraps the base layer about the wound, with the adhesive holding the bandage to the skin. The pad can help to absorb any fluids emanating from the wound. The adhesive layer helps to retain the pad in place atop the wound. One function of the dressing is to prevent infection of the wound. The dressing provides a barrier to materials that might contaminate the wound, including contaminating liquids or bacteria.

A problem with prior art bandages is that the pad can adhere to the wound when exudate flows from the wound to the pad and dries. Consequently, when a user attempts to remove the bandage, the adhered pad risks tearing and re-opening the wound, thus delaying recovery and causing unnecessary pain. It would be advantageous to have an improved bandage.

### BRIEF DESCRIPTION OF THE DRAWINGS

FIG. 1 illustrates an exploded view of one explanatory bandage in accordance with one or more embodiments of the disclosure.

FIG. 2 illustrates a first perspective view of one explanatory bandage in accordance with one or more embodiments of the disclosure.

FIG. 3 illustrates a second perspective view of one explanatory bandage in accordance with one or more embodiments of the disclosure.

FIG. 4 illustrates a first plan view of one explanatory bandage in accordance with one or more embodiments of the disclosure.

FIG. 5 illustrates a second plan view of one explanatory bandage in accordance with one or more embodiments of the disclosure.

FIG. 6 illustrates an exploded view of another explanatory bandage in accordance with one or more embodiments of the disclosure.

FIG. 7 illustrates various explanatory bandages in accordance with one or more embodiments of the disclosure.

FIG. 8 illustrates a pad of a bandage in accordance with one or more embodiments of the disclosure becoming wet from exudate or another wetting agent.

FIG. 9 illustrates a pad of a bandage in accordance with one or more embodiments of the disclosure releasing from a film layer after becoming wet from exudate or another wetting agent.

FIG. 10 illustrates one step of a method of using one explanatory bandage in accordance with one or more embodiments of the disclosure.

FIG. 11 illustrates another step of a method of using one explanatory bandage in accordance with one or more embodiments of the disclosure.

FIG. 12 illustrates another step of a method of using one explanatory bandage in accordance with one or more embodiments of the disclosure.

FIG. 13 illustrates one explanatory method of manufacturing a bandage in accordance with one or more embodiments of the disclosure.

FIG. 14 illustrates how components of one explanatory bandage can be color-coded in accordance with one or more embodiments of the disclosure.

FIG. 15 illustrates one explanatory backing layer in accordance with one or more embodiments of the disclosure.

FIG. 16 illustrates another explanatory backing layer in accordance with one or more embodiments of the disclosure.

FIG. 17 illustrates a bottom plan view of one explanatory bandage configured in accordance with one or more embodiments of the disclosure.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present disclosure.

### DETAILED DESCRIPTION OF THE DRAWINGS

Embodiments of the disclosure are now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.” Relational terms such as first and second, top and bottom, and the like may be used solely to distinguish one entity or action from another entity or action without necessarily requiring or implying any actual such relationship or order between such entities or actions. Also, reference designators shown herein in parenthesis indicate components shown in a figure other than the one in discussion. For example, talking about a device (10) while discussing figure A would refer to an element, 10, shown in figure other than figure A.

Embodiments of the disclosure provide a bandage comprising a backing layer, a film layer, and an absorbent layer. The film layer, which may be pellucid, defines a first major face and a second major face. In one embodiment, one or more design elements are printed or otherwise disposed along the first major face. The design elements can provide an indicator that the bandage is of a specific type, such as one that is “truly ouchless” or that will release the absorbent pad when it absorbs a wetting agent such as wound exudate.

In one embodiment, an adhesive layer is disposed along the second major face of the film layer. The adhesive layer can be continuously disposed along the film layer or discontinuously disposed along the film layer. The adhesive layer can then be attached to the adhesive layer at an interface or to the film layer directly by way of a second adhesive layer at an interface. In one or more embodiments, the interface is to release the absorbent layer when the absorbent layer absorbs a wetting agent. Advantageously, this allows the backing layer and/or film layer to be removed while leaving the absorbent layer attached to a wound when exudate or other moisture has caused adhesion between the adhesive layer and the wound. Embodiments of the disclosure thus advantageously work to cause release of the absorbent pad from the backing layer and/or film layer to prevent wound healing disruption or irritation when the absorbent pad has become attached to the wound.

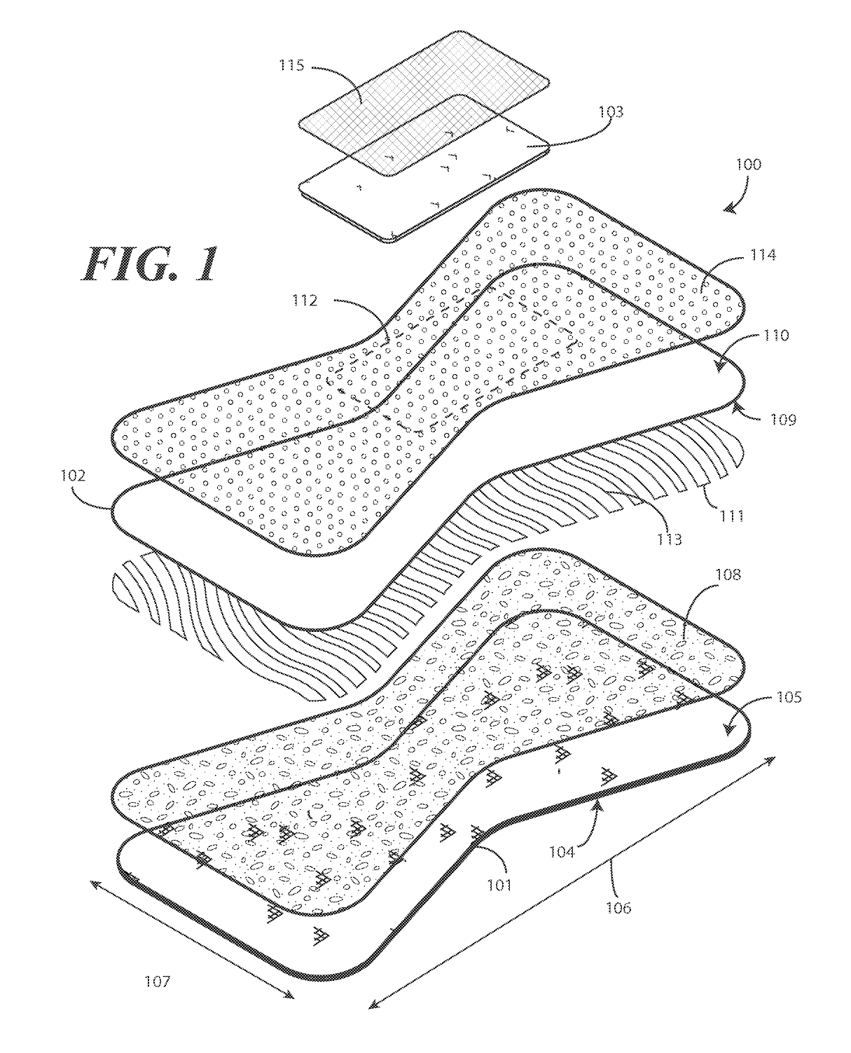

Turning now to FIG. 1, illustrated therein is an exploded view of one explanatory bandage 100 configured in accordance with one or more embodiments of the disclosure. In one embodiment, the basic structural elements of the bandage 100 include a backing layer 101, a film layer 102, and an absorbent layer 103. Other optional components can be included as well as will be noted in the discussion below.

The backing layer 101 can be manufactured from a variety of materials. In one embodiment, the backing layer 101 is manufactured from a thermoplastic material. In other embodiments, different materials can be used. The backing layer 101 can be manufactured from various materials or fabrics suitable for use in bandage applications. In one embodiment, the backing layer 101 and is pliant and at least slightly stretchable. In other embodiments, the backing layer 101 can be semi-flexible and/or rigid. In one embodiment, the backing layer 101 is manufactured from a spun polyester fabric. Such a material is suitable for use as the backing layer 101 because it is breathable and provides a wicking capability for stray fluids.

Those of ordinary skill in the art having the benefit of this disclosure will appreciate that spun polyester is but one type of material suitable for the backing layer 101. Plastic-based materials, rubber-based materials, elastic fabrics, foams, flex-fabrics, sheer fabrics, paper, or other materials may also be used. For example, in one embodiment, the backing layer 101 is manufactured form a monolithic film. Additionally, various woven, non-woven, hydroentangled materials, and/or combinations thereof, absorbent Airlaid, spunlace, blends of polyester, polypropylene, polyethylene, urethane, and/or combinations thereof can be used. These materials can be manufactured using various methods, including a spunbond meltblown spundbond method, a spunbond meltblown meltblown spundbond method, and a spunbond meltblown meltblown spundbond method. In other embodiments, the backing layer 101 can be manufactured from two layers that are coupled together. A first layer can comprise, for example, a blue thermo-polyehtylene (TPE) film that is covered in adhesive. Atop the TPE layer is disposed another layer, which may be manufactured from Sontara®, which is manufactured by the DuPont Corporation.

In another embodiment, the backing layer 101 is manufactured from a woven fabric. Other materials, such as perforated films, non-woven fabrics, foams, or other materials can be used as the backing layer 101 in one or more embodiments. Plasticized polymers such as polyurethane, polypropylene, or polyethylene can be used as well.

In one embodiment the backing layer 101 is manufactured from TPE. Alternatively, a thermoplastic polyurethane (TPU) can be used. The TPU or TPE can be configured to be breathable via the inclusion of micropores that allow vapor to penetrate the TPU or TPE while precluding liquids from passing through the same. While other materials—be they breathable or not—can be used, vapor penetrable backing material can help wounds heal more quickly. Breathable TPU and TPE materials are available from companies such as American Polyfilm, Inc., of Brandford, Conn. Breathable TPU or TPE is well suited as the backing layer 101 due to its high durability, high abrasion resistance, and low-temperature flexibility. In one embodiment, the backing layers 101 are manufactured from textured, breathable TPU or TPE having an adhesive layer disposed along a patient side of the backing layer 101.

In one embodiment, the backing layer 101 defines a first major face 104 and a second major face 105, disposed on a side of the backing layer 101 opposite to the first major face 104. The second major face 105 is shown in more detail with reference to FIG. 3 below.

In one embodiment, the backing layer 101 is opaque. In another embodiment, the backing layer 101 pellucid. Where the backing layer 101 is opaque, it can be manufactured from a colored material in one or more embodiments. A colored coating can be applied to the backing layer 101 as well.

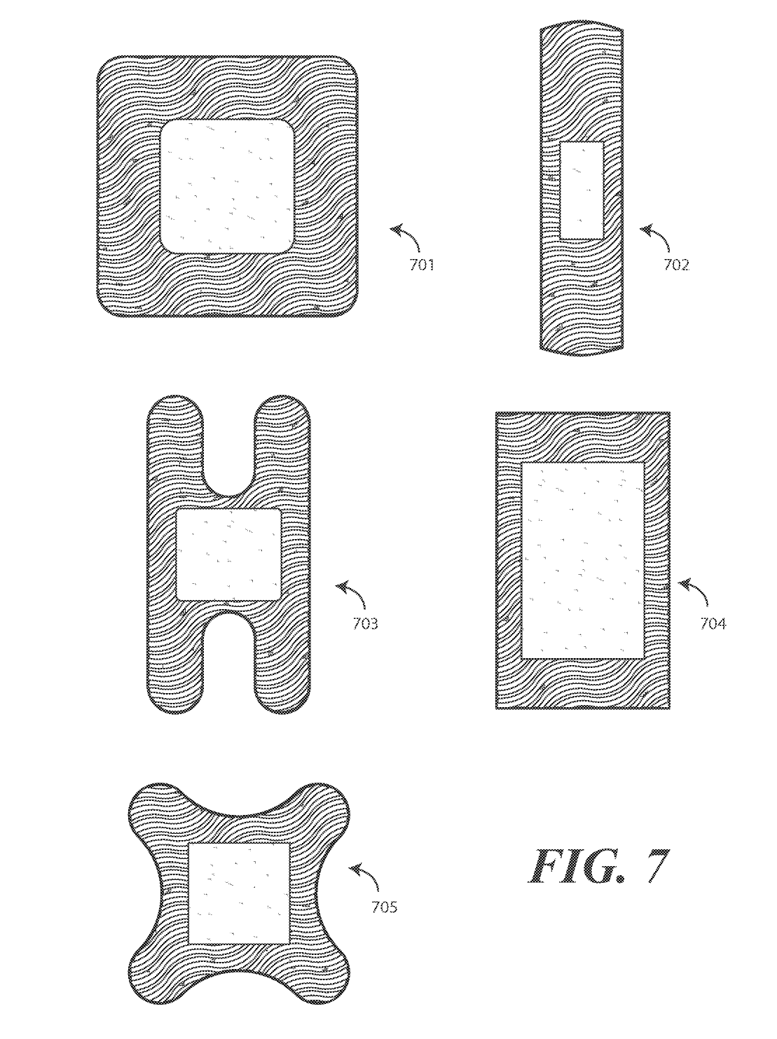

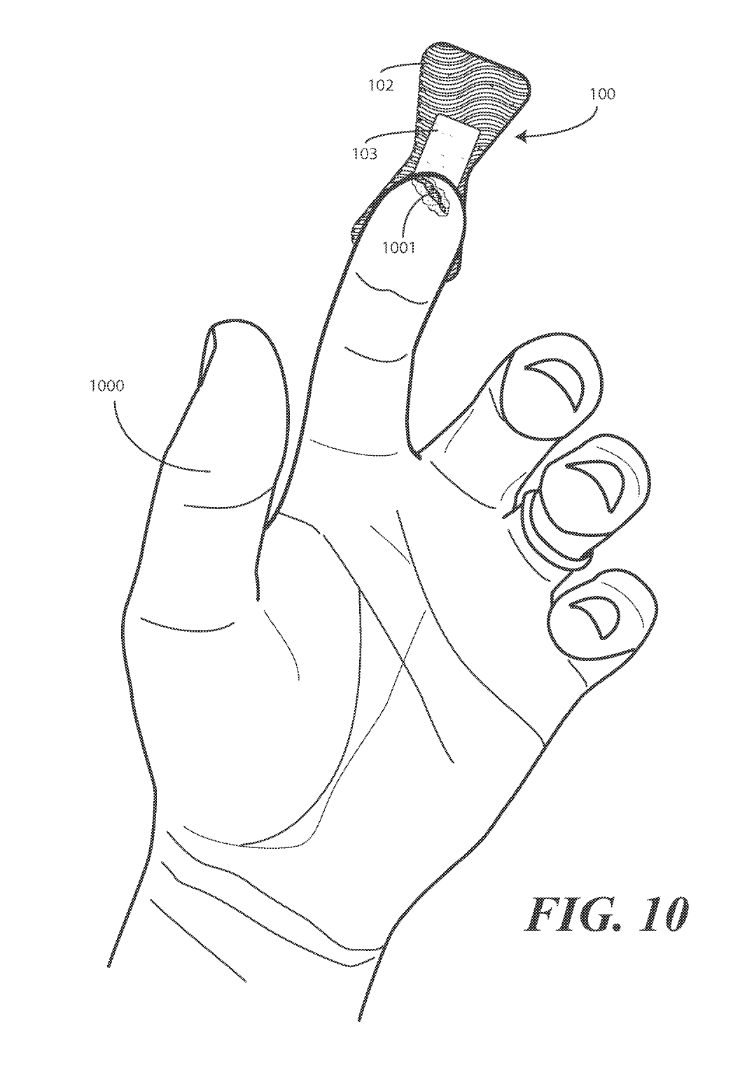

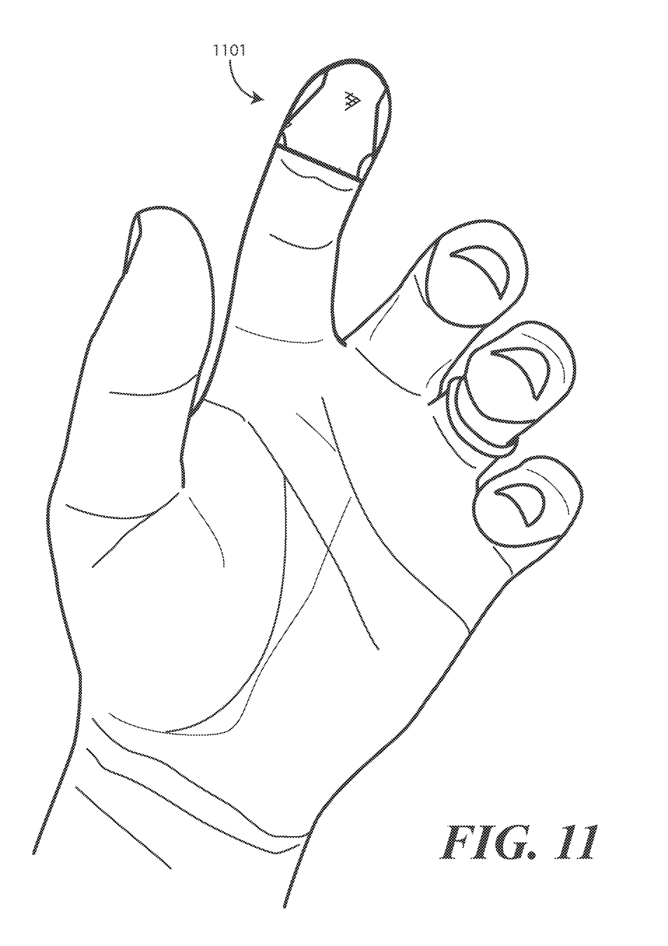

While the backing layer 101 of FIG. 1 is generally shaped like a bow-tie to facilitate adhesion to a finger as will be shown below with reference to FIGS. 10-12, it will be obvious to those of ordinary skill in the art having the benefit of this disclosure that the backing layer can take other shapes as well. A few alternate shapes will be shown below with reference to FIG. 7. For example, in on embodiment the backing layer 101 is rectangular. In other embodiments the backing layer 101 can be shaped to accommodate a particular need or application. The backing layer 101 can be cut or shaped into any number of design shapes, silhouette shapes, or other desired shapes.

In one embodiment, a length 106 of the backing layer 101 is between one and two inches, inclusive. In one embodiment, a width 107 of the backing layer 101 is between one-half and one inches, inclusive. Other dimensions will be obvious to those of ordinary skill in the art having the benefit of this disclosure.

In one or more embodiments, the backing layer 101 is perforated. The perforation can be achieved by punching holes through a thermoplastic material, by using a woven or non-woven material as the backing layer 101, or by other methods. This allows air to pass from a side adjacent to the first major face 104 to the opposite side adjacent to the second major face 105. Perforating the backing layer 101 can allow ambient air to reach a wound, which is some cases, can result in faster healing.

In one or more embodiments, an adhesive layer 108 is disposed along the second major face 105 of the backing layer 101. The adhesive layer 108 can be rolled, sprayed, vapor deposited, or otherwise deposited on the second major face 105 of the backing layer 101. The adhesive layer 108 can be initially placed on the backing layer 101. The film layer 102 can then be placed along the adhesive layer 114. In one embodiment, the adhesive layer 108 comprises a pressure sensitive adhesive. Other types of adhesive will be obvious to those of ordinary skill having the benefit of this disclosure.

In one embodiment, the adhesive deposited on the second major face 105 of the backing layer 101 is a medical grade adhesive manufactured by companies such as Hollister or 3M. One example includes Scotch-Weld™ adhesive manufactured by 3M Corporation. Another is cynoacrolate, which is manufactured by a variety of manufacturers. The adhesive used for the adhesive layer 108 can be the same adhesive, or can be a different adhesive.

The film layer 102, like the backing layer, can be manufactured from a variety of materials. In one embodiment, the film layer 102 is manufactured from TPE. Alternatively, TPU can be used. The TPU or TPE can be configured to be breathable via the inclusion of micropores that allow vapor to penetrate the TPU or TPE while precluding liquids from passing through the same. While other materials—be they breathable or not—can be used, vapor penetrable film layer 102 can help wounds heal more quickly, as noted above. Other materials, including foam, sheer materials, or paper can also be used as the film layer 102.

In one illustrative embodiment, the film layer 102 is formed by a single piece of clear, breathable, TPE or TPU film. While the film layer 102 is adhesively coupled to the backing layer 101 in this illustrative embodiment, they could be thermally bonded together or coupled together by other methods as well. In one embodiment, the film layer 102 and is pliant and at least slightly stretchable. In other embodiments, the film layer 102 can be semi-flexible and/or rigid.

As with the backing layer 101, the film layer 102 defines a first major face 109 and a second major face 110. In one or more embodiments, the film layer 102 is manufactured from a material that supports ink or other pigment printing processes to carry one or more design elements 111 disposed on one of the first major face 109 or the second major face 110.

In the illustrative embodiment of FIG. 1, the first major face 109 carries one or more design elements 111. The design elements 111 can provide a form of decoration, graphic, image, or other ornamentation along a major face of the film layer 102. While the design elements 111 can take many forms, in one or more embodiments of the disclosure the design elements 111 define a graphical pattern as shown in FIG. 1. The graphical pattern can comprise an identifier indicating an interface 112 defined between the film layer 102 and the absorbent layer 103 is to release the absorbent layer 103 when the absorbent layer 103 absorbs a wetting agent, as will be described in more detail below. In the illustrative embodiment of FIG. 1, the graphical pattern comprises a plurality of swirls 113. Other graphic patterns will be obvious to those of ordinary skill in the art having the benefit of this disclosure.

The design elements 111 can be disposed along the major face of the film layer 102 by depositing colorants via a printing process or by other techniques. For example, the design elements 111 can comprises a printable material that is suitable for deposition along the film layer 102 from a printer, such as an ink jet printer, laser printer, or other type of printer, to form graphic patterns along the film layer 102. The design elements 111 can be constructed from a covering disposed along a major face of the film layer 102 as well. As noted above, in one embodiment the film layer 102 is transparent, clear, and/or pellucid so that the design elements 111 can be seen through the film layer 102.

In one or more embodiments, the film layer 102 can be configured in a sheet with each film layer section separable from another via perforations disposed along perimeters of the film layer sections. The sheet can then feed into a printer that is operable with a controller and a database that stores templates and/or design elements. The controller, which can be a computer, processor, or other circuitry, retrieves the templates and/or design elements from the database and applies them to the film layer sections via the printer. In one embodiment, the printer is an ink jet printer and the design elements comprise ink that is deposited along each film layer 102. However, other types of printers suitable for depositing the design elements 111 on the film layer 102 will be obvious to those of ordinary skill in the art having the benefit of this disclosure. Once the design elements 111 are created, the printer ejects the sheet for assembly and packaging.

In one embodiment, an adhesive layer 114 is disposed along the second major face 110 of the film layer 102. In the illustrative embodiment of FIG. 1, the adhesive layer 114 is continuous across the second major face 110 of the film layer 102. As with adhesive layer 108, adhesive layer 114 can be rolled, sprayed, vapor deposited, or otherwise deposited on the second major face 110 of the film layer 102.

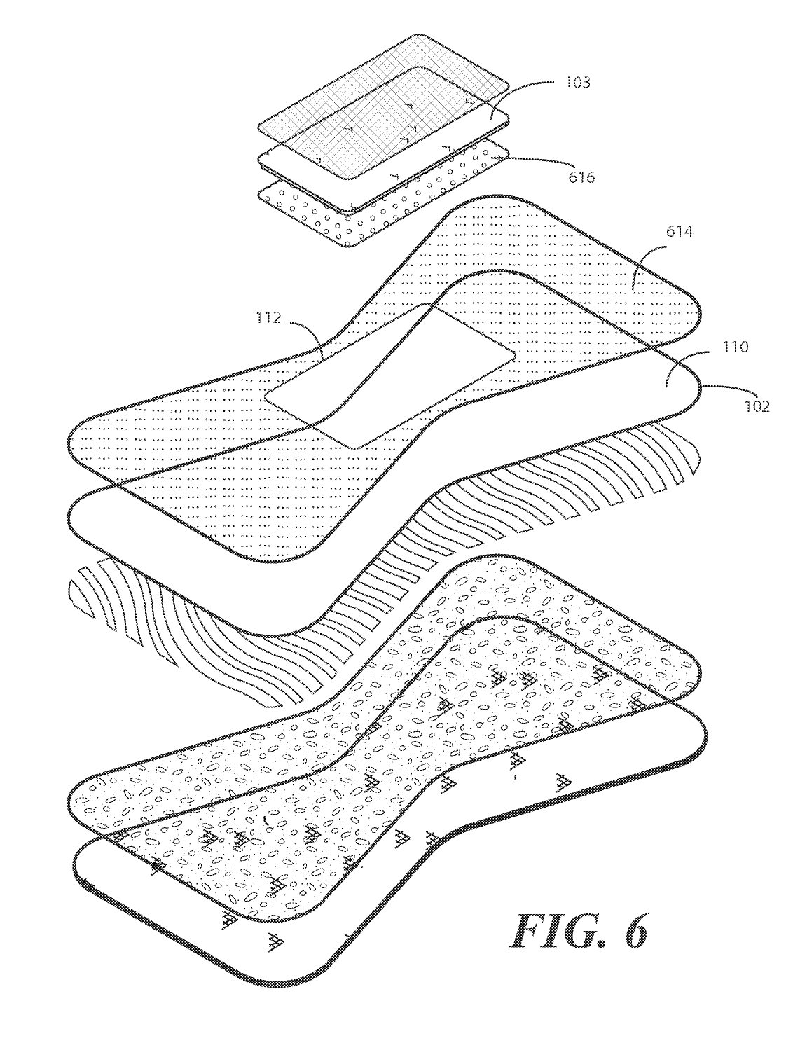

In one embodiment, the adhesive layer 114 deposited on the second major face 110 of the film layer 102 comprises a hydrophobic adhesive. For example, in one embodiment the hydrophobic adhesive comprises a silicone-based adhesive. In other embodiments, the adhesive layer 114 deposited on the second major face 110 of the film layer 102 comprises a hydrophyllic adhesive. The adhesive layer 114 can be deposited along all of the second major face 110 of the film layer 102 in one embodiment. Alternatively, the adhesive layer 114 can be selectively deposited only along portions of the second major face 110 of the film layer 102 as will be shown in FIG. 6 below.

In one embodiment, the absorbent layer 103 is disposed along the second major face 110 of the film layer at an interface 112. In one embodiment, the absorbent layer 103 comprises a non-woven absorbent pad. For example, the absorbent pad can be manufactured from a non-woven gauze material, such as 5 oz. gauze, cotton felt material, or other similar non-woven material. Other materials, such as polyester, polyester fiber, alginates, foams, rayon/polyester blends, and so forth can also be used. Combinations of absorptive materials could also be used.

Anti-microbial, antibiotic, and anti-bacterial substances can be added to the absorbent layer 103 as well. For example, in one or more embodiments active ingredients or other compounds to help heal and protect a wound can be integrated into the absorbent layer 103 for release into the wound during bandage usage. A few examples of active ingredients include antimicrobial agents, antibiotics, anti-inflammatory ingredients, vitamins, steroids, skin moisteners, drying agents, and so forth. Other examples of compounds include honey, tea tree oil, colloidal oatmeal, baking soda, and hemostatic agents. In one embodiment, a benzalkonium chloride (BZK) solution is coated on the absorbent layer 103 by way of a dip and squeeze method. The absorbent layer 103 can be pulled through a vessel where it is immersed in the BZK solution, and then passes through a nip roller to squeeze out excess BZK solution. The absorbent layer 103 can then pulled through a long drying oven.

It should be noted that BZK is a very unique active ingredient for incorporation into the absorbent layer 103. BZK is different from, for example, antibiotics because BZK operates as an antimicrobial agent within the absorbent layer 103, while antibiotics must exit the absorbent layer 103 to operate on a wound itself. Thus, in one or more embodiments the active ingredient incorporated with the absorbent layer 103 is to operate on the absorbent layer 103 and not on a wound to which the bandage 100 is applied.

In the world of drugs, the term antibiotic is thought to be broader in scope than antibacterial. Those of ordinary skill in the art appreciate that “antibacterial” serves as a subclass of antibiotic, which means antibacterial is directed to strictly killing bacteria while antibiotic could kill yeast and fungus in addition to bacteria.

When it comes to bandages, antibacterial compounds and antibiotic compounds are not technically directly comparable. Adding an antibacterial compound to the absorbent layer 103 speaks to the compounds in the absorbent layer 103 that kill bacteria. By contrast, antibiotic compounds kill bacteria and possibly other organisms inside the body only after they exit the absorbent layer 103 to interact with the wound.

With this in mind, the differences between the inclusion of antibacterial compounds to the absorbent layer 103 and antibiotic compounds to the absorbent layer 103 can be underscored. Some prior art devices use antibiotic compounds. By contrast, embodiments of the present disclosure employ antimicrobial or antibacterial compounds. Comparing the two is like comparing apples to oranges. With antibiotic compounds, such compounds are for killing organisms in the wound itself. By contrast, antibacterial compounds are for killing organisms in the absorbent layer 103 itself. Antibacterial compounds function as a barrier to microorganisms. Antibiotic compounds do not.

Moreover, the manufacturing processes for each compound is different. Antibacterial compounds can be incorporated into a dry absorbent layer 103. By contrast, antibiotic compounds are added to an absorbent layer only when it is wet.

Additionally, testing for some antibacterial compounds, such as BZK, can be very different from other antibacterial compounds. For instance, some prior art antibacterial compounds that may have received Food and Drug Administration (FDA) approval a long time ago did so when the FDA has less strict rules on making antibacterial claims. Since the FDA rarely ever overturns a decision made in the past for this type of thing, prior art products get granfathered in when regulationsn change.

For antibacterial products, the larger the log reduction in microorganisms the better. Log reduction means number of bacterial organisms killed, so larger the better. Prior art compounds were only required to meet a “2 log” standard. However, modern devices are required to meet a “4 log” standard due to increased requirements. The use of antimicrobial compounds such as BZK advantageously meets this requirement while prior art compounds do not. For this reason, the use of BZK offers a high standard of efficacy.

The absorbent layer 103 can be manufactured from a patterned layering in one or more embodiments. In other embodiments a pad can be used in place of the absorbent layer 103 that is a non-absorbent pad or that is otherwise hydrophobic. Other types of pads will be obvious to those of ordinary skill in the art having the benefit of this disclosure. In one or more embodiments, the absorbent layer 103 is to collect and hold exudate from a wound.

In one or more embodiments, the interface 112 is to release the absorbent layer when the absorbent layer 103 absorbs a wetting agent. Testing has shown that when a woven absorbent pad is used as the absorbent layer 103 and a silicone-based adhesive is used as the adhesive layer 114 along the interface 112, the absorbent layer 103 will release from the interface when the absorbent layer 103 absorbs a wetting agent. Advantageously, this results in a truly “ouchless” bandage 100 in which a non-woven absorbent layer 103 is attached to the film layer 102 with a silicone-based adhesive layer 114 at the interface 112 facilitates a causal release of the absorbent layer 103 from the interface 112 when the absorbent layer 103 absorbs a wetting agent such as wound exudate. This release precludes wound disruption or irritation when the bandage 100 is removed because the absorbent layer 103 stays attached to the wound and releases from the film layer 102. Accordingly, in one or more embodiments the interface 112 is designed for intentional separation between the absorbent layer 103 and the film layer 102 as the interface 112 so that any wound attached to the absorbent layer 103 remains uninterrupted. The absorbent layer 103 can thus be left in place so as not to disrupt the wound for proper healing. This facilitates a two-step removal process on heavily draining wounds. The silicone-based adhesive being disposed along the interface 112 allows the bandage 100 to form an occlusive, waterproof dressing in one or more embodiments that protects a wound from outside contaminates and the patient from wound disruption due to exudate strikethrough.

In one or more embodiments, an optional mesh layer 115 can be integrated with, or disposed above, the absorbent layer 103. In one or more embodiments, the mesh layer 115 comprises a porous covering material that spans the absorbent layer 103 on a side of the absorbent layer 103 opposite the interface 112. The mesh layer 115 may be manufactured from a porous polyethylene film, such as high-density polyethylene, a porous polyethylene net, or mesh. It will be obvious to those of ordinary skill in the art having the benefit of this disclosure that other materials for the mesh layer 115 can be used as well. For example, in other embodiments the mesh layer 115 may be manufactured from polyvinyl chloride, nylon, polyester, polypropylene, or other materials.

In one or more embodiments, one or both of the absorbent layer 103 or the mesh layer 115 can be color-coded with a food-grade colorant. In one embodiment, the color-coding is achieved by coating one or both of the absorbent layer 103 or the mesh layer 115 with a non-stick layer that comprises a food-grade, Federal Drug Administration approved, colorant. For example, in one embodiment one or both of the absorbent layer 103 and the mesh layer are configured so as to be visibly distinct from any of the film layer 102, the design elements 111, the adhesive layer 114, or the backing layer 101. This can be achieved by coloring either or both of the absorbent layer 103 or the mesh layer 115 differently from any of the film layer 102, the design elements 111, or the backing layer 101. Colorant can be added to the mesh layer 115 by the introduction of color from pellets that are melted and blended with the polyethylene or other material defining the mesh layer 115 during an extrusion process. In other embodiments, the colorant can be applied as a coating to either or both of the mesh layer 115 or the absorbent layer 103. In other embodiments, the colorant can be integrated into one or both of the mesh layer 115 or the absorbent layer 103. In other embodiments.

Illustrating by example, either or both of the absorbent layer 103 or the mesh layer 115 can be blue, while the other elements are a different color such as tan, khaki, taupe, white, green, or another color. In one or more embodiments, either or both of the absorbent layer 103 or the mesh layer 115 is colored or impregnated with a food-grade colorant so as not to impact healing of any wound that contacts either or both of the absorbent layer 103 or the mesh layer 115. Other examples if visible distinctions will be obvious to those of ordinary skill in the art having the benefit of this disclosure.

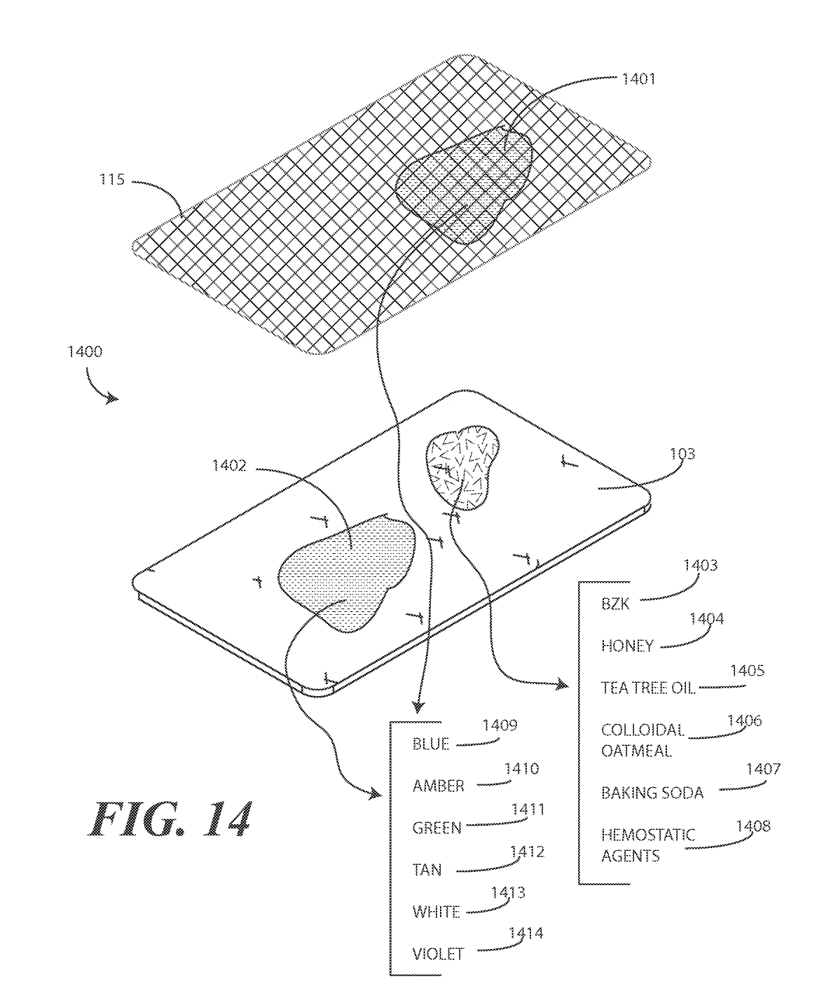

Turning now briefly to FIG. 14, illustrated therein are the mesh layer 115 and the absorbent layer 103 after each has been color-coded with a colorant 1401,1402. In one embodiment, the mesh layer 115 is color-coded with a colorant 1401, while the absorbent layer 103 is not color-coded. In another embodiment, the mesh layer 115 is not color-coded, while the absorbent layer 103 is color-coded with a colorant 1402. In yet another embodiment, both the mesh layer 115 and the absorbent layer 103 are color-coded with colorants 1401,1402.

Where the latter occurs, in one embodiment each colorant 1401,1402 is the same color. For example, both colorant 1401 and colorant 1402 can be yellow in one embodiment. In another embodiment, each colorant 1401,1402 can be a different color. For example, in one embodiment colorant 1401 is blue while colorant 1402 is yellow. This can five the overall assembly 1400 comprising the mesh layer 115 and the absorbent layer 103 a visible appearance of green. Other color combinations will be obvious to those of ordinary skill in the art having the benefit of this disclosure.

As noted above, in one or more embodiments, compounds, anti-microbial, antibiotic, and anti-bacterial substances can be added to the absorbent layer 103. These active ingredients can help heal and protect a wound can be integrated into the absorbent layer 103 for release into the wound during bandage usage. In the illustrative embodiment of FIG. 14, BZK 1403 has been added to the absorbent layer 103. Other active ingredients will be obvious to those of ordinary skill in the art having the benefit of this disclosure.

For example, in another embodiment, honey 1404 is integrated with the absorbent layer 103. In another embodiment, tea tree oil 1405 is integrated with the absorbent layer 103. In still another embodiment, colloidal oatmeal 1406 is integrated with the absorbent layer 103. Baking soda 1407 or hemostatic agents 1408 can be integrated into the absorbent layer 103 as well. Accordingly, in one or more embodiments, a compound selected from a plurality of compounds, such as BZK 1403, honey 1404, tea tree oil 1405, colloidal oatmeal 1406, baking soda 1407, or hemostatic agents 1408, can be integrated with the absorbent layer 103.

In one embodiment, the colorant 1401,1402 applied to any of the mesh layer 115, the absorbent layer 103, or combinations thereof, can serve as an indicator color-coding. For instance, in one embodiment the mesh layer 115 is colored with a colorant 1402 to indicate that BZK 1403 or another active ingredient has been added to the absorbent layer 103. In another embodiment, the absorbent layer 103 is color-coded with a colorant 1402 to indicate that the absorbent layer 103 includes BZK 1403 or another active ingredient. In one embodiment, the colorant 1402 is colored blue 1409. Of course, combinations of colorants 1401,1402 can be used as previously described. This color-coding indicator allows consumers to quickly realize that the absorbent layer 103 of the corresponding bandage includes an active ingredient. This is in contrast to prior art bandages where any absorbent materials are white.

In one or more embodiments, the mesh layer 115 is color-coded to indicate which of the plurality of compounds is integrated with the absorbent layer 103. For example, where the compound comprises honey 1404, the mesh layer 115 can be color-coded amber 1410. In another embodiment, where the compound comprises tea tree oil 1405, the mesh layer 115 can be color-coded green 1411. In yet another embodiment, the compound can comprise colloidal oatmeal 1406, and the mesh layer 115 can be color-coded tan 1412. In yet another embodiment, where the compound comprises baking soda 1407, the mesh layer 1415 can be color-coded white. In yet another embodiment, where the compound comprises a hemostatic agent 1408, the mesh layer 115 can be color-coded violet 1414. These examples are illustrative only, as other color codings will be obvious to those of ordinary skill in the art having the benefit of this disclosure. It should be noted that while the mesh layer 115 was described as having these color coding schemes in one embodiment, the absorbent layer 103 could have the coloring agents as well.

Turning now back to FIG. 1, in one or more embodiments, one or more releasable liners (not shown) can be attached along the adhesive layer 114 so as to prevent the adhesive layer 114 from sticking to anything prior to usage. A user can remove the releasable strips to expose the adhesive layer 114. The user can then place the absorbent layer 103 over a wound and press the first major face 104 of the backing layer 101 to apply the bandage 100 to a wound. The one or more releasable strips can comprise wax release backing paper to keep the adhesive layer 114 from sticking to itself or other objects in packaging.

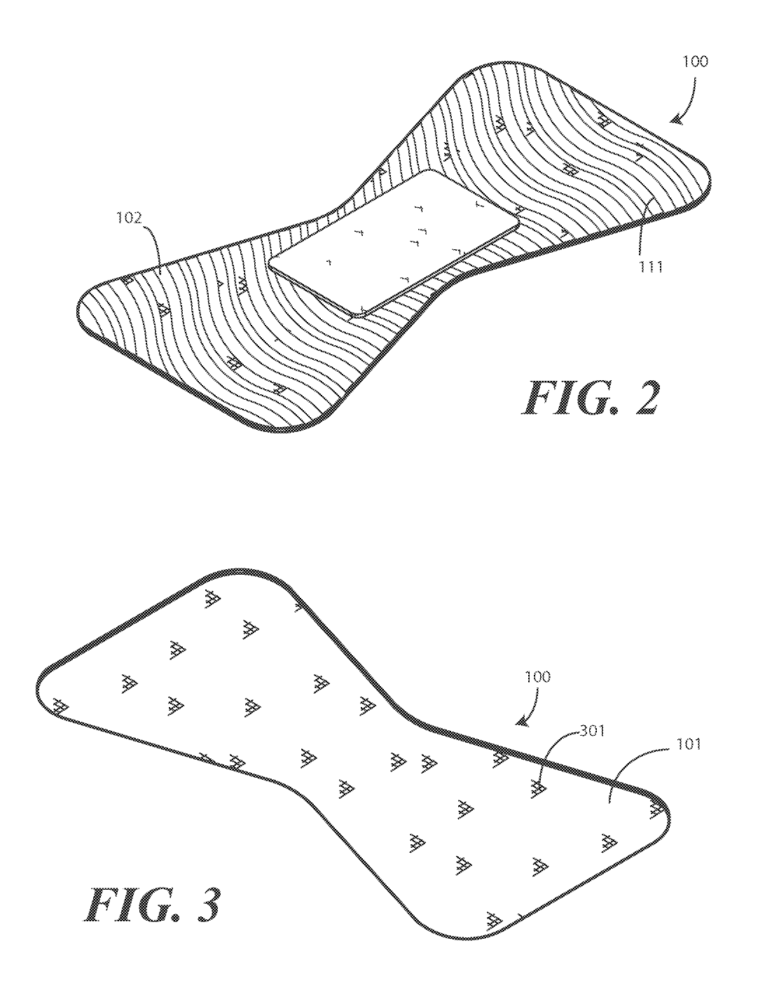

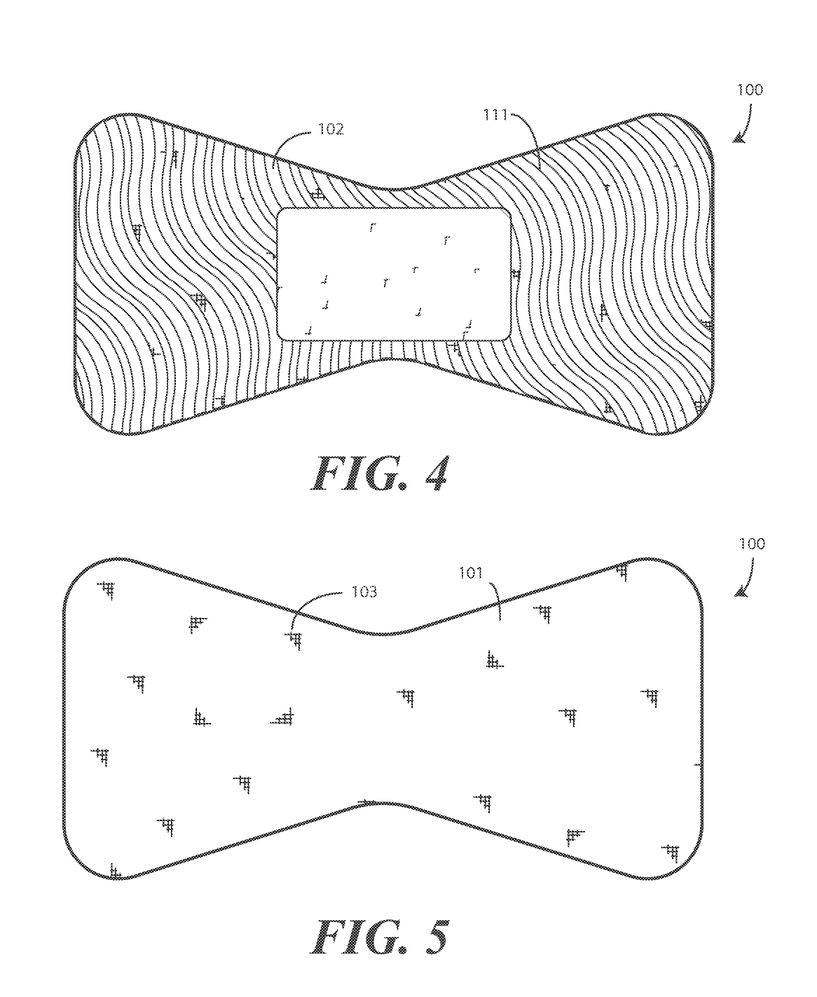

Once the various layers are put together, the resulting bandage 100 is shown in FIGS. 2-5. FIG. 2 illustrates a first perspective view of the bandage 100, while FIG. 3 illustrates a second perspective view of the bandage 100. FIGS. 4-5 illustrate a bottom plan view and a top plan view of the bandage 100, respectively. FIGS. 2 and 4 show how the design elements 111 are visible through the film layer 102 in one or more embodiments, while FIGS. 3 and 5 illustrated ornamental weave components 301 in the backing layer 101 when a non-woven fabric is used as the backing layer 101.

Turning now to FIG. 6, in one or more embodiments the adhesive layer 614 can be discontinuous across the second major face 110 of the film layer 102. For example, in FIG. 6 the adhesive layer 614 is discontinuous under the absorbent layer 103. Where this is the case, a second adhesive layer 616 can be disposed along the interface 112 between the absorbent layer 103 and the film layer 102. As noted above, in one or more embodiments, using a silicone-based adhesive along the interface 112 allows the absorbent layer 103 to release from the film layer 102 to protect the patient from wound disruption due to exudate strikethrough. Accordingly, in one or more embodiments the second adhesive layer 616 comprises a silicone-based adhesive.

To provide extra adhesion to a person's skin about the wound site, in one embodiment the adhesive layer 614 can have a greater adhesion coefficient or “tack” than the adhesive used for the second adhesive layer 616. Adhesion coefficient is a measurement of the stickiness of the adhesive. One way of measuring the adhesion coefficient is by determining what amount of weight can be supported by a square centimeter of the adhesive material. Where a square inch of the material is capable of supporting a greater weight, it will have a greater adhesion coefficient, and vice versa. In one or more embodiments, the adhesion coefficient of the adhesive layer 614 is greater than that of the second adhesive layer 616. For example, in one embodiment the adhesive used along the adhesive layer 614 is an acrylic-based adhesive while the adhesive used along the second adhesive layer 616 is a silicone-based adhesive.

The adhesive layer 614 can be discontinuous in other ways as well. Similarly, the second adhesive layer 616 can be discontinuous as well. For example, in one embodiment one or both of the adhesive layer 614 or the second adhesive layer 616 formed by selectively printing a plurality of adhesive “islands” along the film layer 102 and/or the interface 112. A selective printing process can form the adhesive islands where the islands are printed only portions of the film layer 102 and/or the interface 112. Alternatively, the islands can be placed on the film layer 102 and/or the interface 112 by selective application from a web-fed rolling process where the islands are thermally or otherwise deposited from a carrier material during a pressing process.

As noted above, while the embodiment of FIGS. 1-6 is a bow-tie shape to facilitate adhesion to a finger, it will be obvious to those of ordinary skill in the art having the benefit of this disclosure that the backing layer can take other shapes as well. Turning now to FIG. 7, illustrated therein are a few alternate shapes illustrating examples of the numerous forms in which bandages configured in accordance with the present invention can be configured.

For example, bandage 701 is generally square in shape, while bandage 702 is generally elongated rectangular. Bandage 703 is “H-shaped” to accommodate a particular need or application, while bandage 704 is generally broad rectangular. Bandage 705 is a curvaceous X-shape. These shapes are examples only, as many others will be obvious to those of ordinary skill in the art having the benefit of this disclosure. Bandages in accordance with embodiments of the disclosure can be formed or cut or shaped into any number of design shapes, silhouette shapes, or other desired shapes.

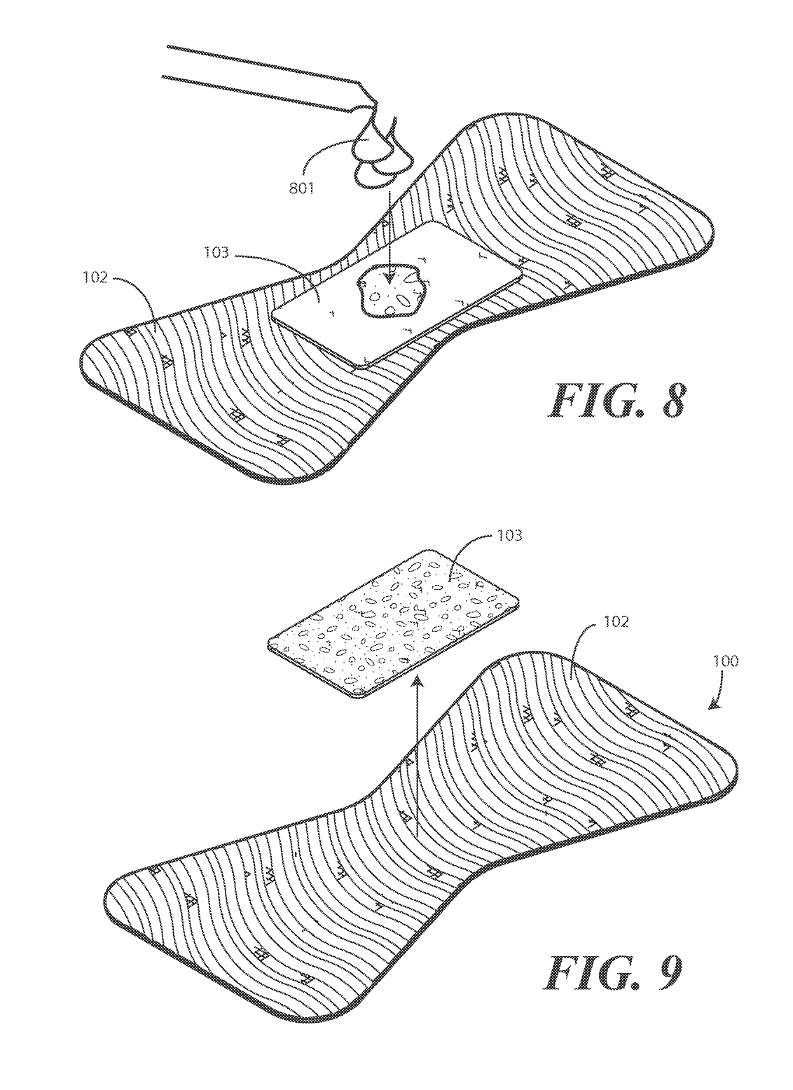

Turning now to FIGS. 8 and 9, illustrated therein is the releasability of the absorbent layer 103 from the film layer 102. Recall from above that in one or more embodiments, the adhesive layer (114) disposed along an interface (112) at the second major face (110) of the film layer 102 is to release the absorbent layer 103 attached thereto when the absorbent layer 103 absorbs a wetting agent 801. In FIG. 8, the absorbent layer 103 is receiving a wetting agent 801, which in one embodiment is wound exudate. As shown in FIG. 9, when a silicone-based adhesive is used as the adhesive layer (114), this facilitates a causal release of the adhesive layer 108 from the film layer 102, which functions to prevent wound interruption when the bandage 100 is removed from a wound to which the absorbent layer 103 has attached due to exudate strikethrough.

Turning now to FIGS. 10-12, illustrated therein is a user 1000 using an explanatory bandage 100 in accordance with one or more embodiments of the disclosure. FIGS. 10-12 illustrate how embodiments of the disclosure offer intentional separation of the absorbent layer 103 from the film layer 102 so that a wound 1001 is not interrupted.

Beginning with FIG. 10, the user 1000 has sustained a wound 1001, which appears to be a minor cut or laceration. Such wounds 1001 are likely to release exudate during the healing process. To protect the wound 1001 from outside debris, contaminants, and pathogens, the user 1000 is applying a bandage 100 configured in accordance with one or more embodiments of the disclosure such that the absorbent layer 103 covers the wound 1001. The resulting bandaged hand 1101 is shown in FIG. 11.

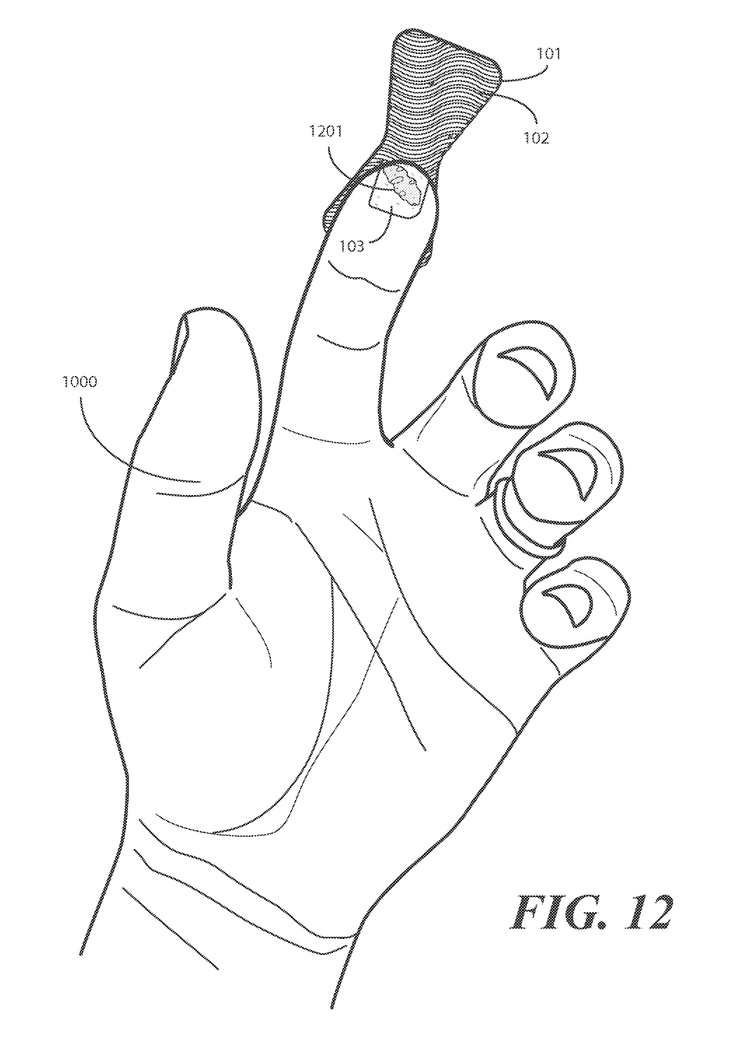

As shown in FIG. 12, the wound (1001) has released exudate 1201 into the absorbent layer 103. Accordingly, when the user 1000 removes the combined backing layer 101 and film layer 102, the absorbent layer 103 releases from the film layer 102 and stays attached to the wound (1001). This results in a two-step removal process in which the combined backing layer 101 and film layer 102 can be removed at any time and the absorbent layer 103 can be removed only once the wound (1001) has healed.

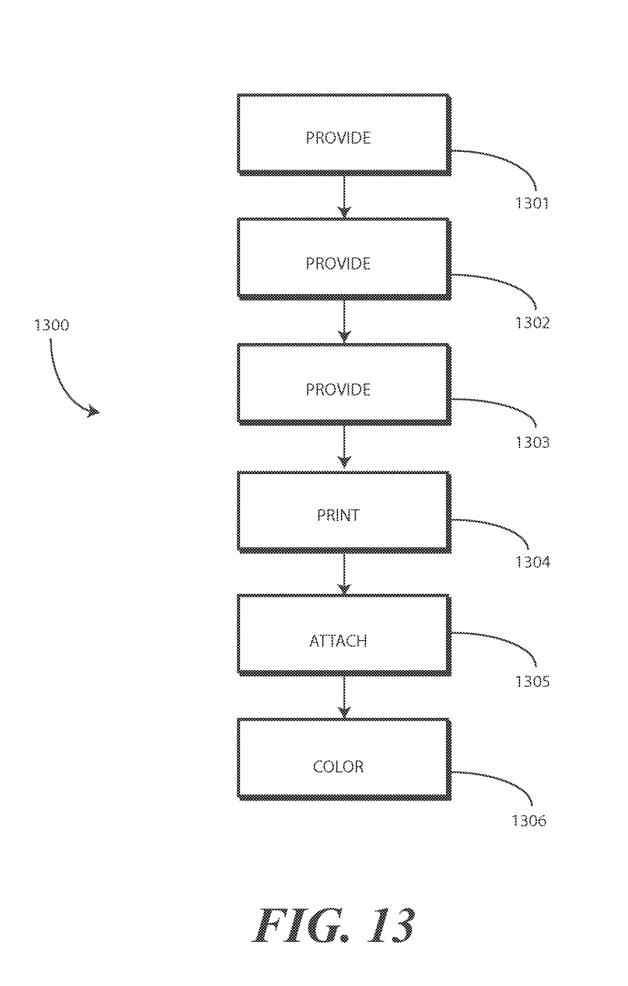

Turning now to FIG. 13, illustrated therein is one explanatory method 1300 of manufacturing a bandage in accordance with one or more embodiments of the disclosure. Many of the steps have been described in detail with reference to FIG. 1 above, and accordingly will only be cursorily discussed here.

At step 1301, the method 1300 provides a backing layer. At step 1302, the method 1300 provides a film layer. At step 1303, the method 1300 provides an absorbent pad.

At step 1304, the method 1300 optionally prints one or more design elements along a first major face of the film layer. At step 1305, the method 1300 disposes an adhesive layer along a second major face of the film layer. In one embodiment, the adhesive layer disposed along an interface between the film layer and the absorbent pad is a silicone-based adhesive. In one embodiment, the adhesive layer is to release an absorbent pad attached thereto when the absorbent pad absorbs a wetting agent. In one embodiment step 1305 also includes attaching the absorbent pad provided at step 1303 to the adhesive layer.

At step 1306, the method 1300 optionally color codes one or both of the absorbent pad or a mesh layer disposed above the absorbent pad to identify the bandage as a truly “ouchless” bandage that will release the absorbent pad if the absorbent pad sticks to a wound. At step 1307, the method 1300 attaching the first major face of the film layer to the backing layer to form a bandage.

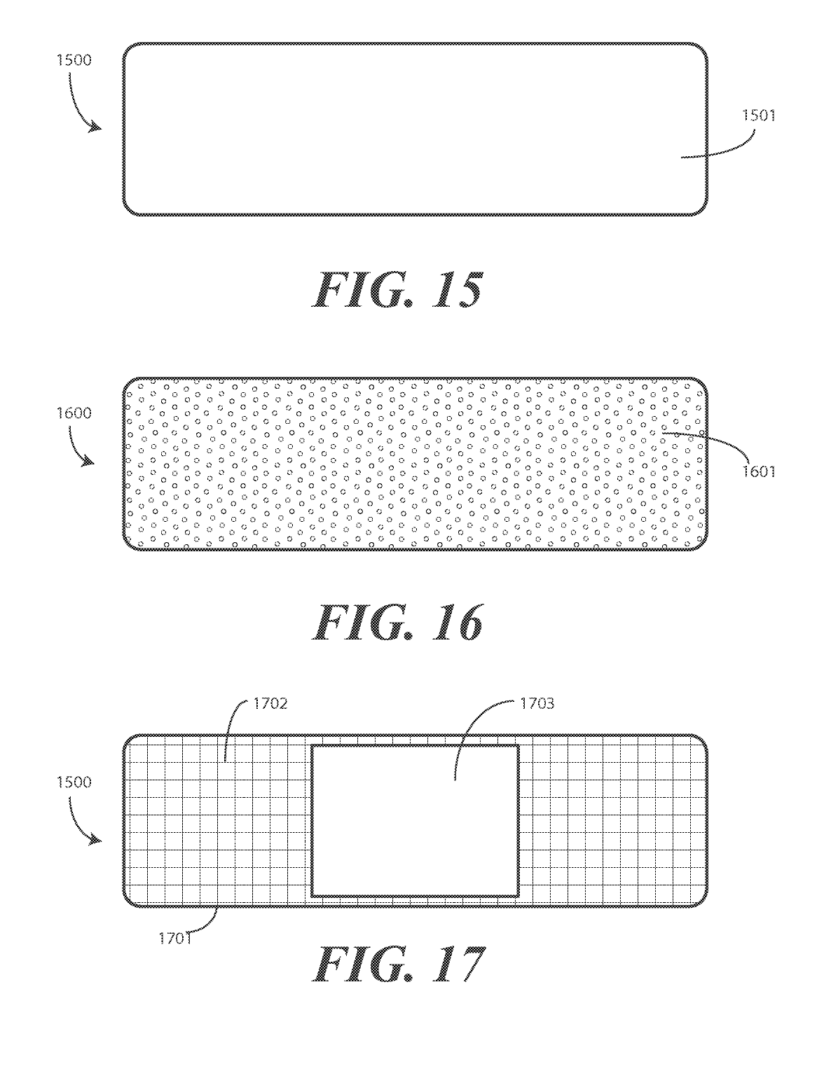

It should be noted that the construction shown in FIGS. 1-7 is but one example of the many ways bandages configured in accordance with one or more embodiments of the disclosure can be constructed. Turning briefly to FIGS. 15-17, illustrated therein is another construction.

Beginning with FIG. 15, illustrated therein in is another backing layer 1500 configured in accordance with one or more embodiments of the disclosure. The backing layer 1500 of FIG. 15 defines a first major face 1501 and a second major face, disposed on a side of the backing layer 1500 opposite to the first major face 1501. In one embodiment, the backing layer 1500 is opaque. In another embodiment, the backing layer 1500 pellucid. Where the backing layer 1500 is opaque, it can be manufactured from a colored material in one or more embodiments.

In one embodiment, the backing layer 1500 is manufactured from a thermoplastic material. In other embodiments, different materials can be used. The backing layer 1500 can be manufactured from various materials or fabrics suitable for use in bandage applications. In one embodiment, the backing layer 1500 and is pliant and at least slightly stretchable. In other embodiments, the backing layer 1500 can be semi-flexible and/or rigid.

Turning now to FIG. 16, illustrated therein is another backing layer 1600 suitable for use with one or more embodiments of the disclosure. The backing layer 1600 of FIG. 16, like the backing layer (1500) of FIG. 15, defines a first major face 1601 and a second major face. The second major face is disposed on a side of the backing layer 1600 opposite to the first major face 1601. The backing layer 1600 can be manufactured from a thermoplastic material that is pliant and at least slightly stretchable.

In the illustrative embodiment of FIG. 16, the backing layer 1600 is perforated. This allows air to pass from a side adjacent to the first major face 1601 to the opposite side adjacent to the second major face. Perforating the backing layer 1600 allows ambient air to reach a wound, which is some cases, can result in faster healing.

Turning now to FIG. 17, illustrated therein is the backing layer 1500 of FIG. 15. In FIG. 17, the second major face 1701 is shown. In one embodiment, the second major face has an adhesive layer 1702 and an absorbent layer 1703 disposed thereon. The adhesive layer 1702 can be rolled, sprayed, vapor deposited, or otherwise deposited on the second major face 1701 of the backing layer 1500. The adhesive layer 1702 can be initially placed on the backing layer 1500. The absorbent layer 1703 can then be placed along the adhesive layer 1702. In this illustrative embodiment, the absorbent layer 1703 is placed centrally along the backing layer 1500. In one embodiment, the adhesive layer 1702 comprises a pressure sensitive adhesive. Other types of adhesive will be obvious to those of ordinary skill having the benefit of this disclosure.

In the foregoing specification, specific embodiments of the present disclosure have been described. However, one of ordinary skill in the art appreciates that various modifications and changes can be made without departing from the scope of the present disclosure as set forth in the claims below. Thus, while preferred embodiments of the disclosure have been illustrated and described, it is clear that the disclosure is not so limited. Numerous modifications, changes, variations, substitutions, and equivalents will occur to those skilled in the art without departing from the spirit and scope of the present disclosure as defined by the following claims. Accordingly, the specification and figures are to be regarded in an illustrative rather than a restrictive sense, and all such modifications are intended to be included within the scope of present disclosure. The benefits, advantages, solutions to problems, and any element(s) that may cause any benefit, advantage, or solution to occur or become more pronounced are not to be construed as a critical, required, or essential features or elements of any or all the claims.

What is claimed is:

1. A bandage, comprising:
a backing layer;
an absorbent layer;
an adhesive layer disposed between the backing layer and the absorbent layer;
an antimicrobial agent integrated with the absorbent layer; and
a mesh layer disposed along the absorbent layer on a side opposite the adhesive layer;
the mesh layer color-coded to indicate the antimicrobial agent is integrated with the absorbent layer.

2. The bandage of claim 1, the antimicrobial agent comprising benzalkonium chloride.

3. The bandage of claim 2, the mesh layer color-coded with a food grade colorant.

4. The bandage of claim 3, the mesh layer color-coded by coating with the food grade colorant.

5. A bandage, comprising:
a backing layer;
an absorbent layer;
an adhesive layer disposed between the backing layer and the absorbent layer;
a compound selected from a plurality of compounds, the compound integrated with the absorbent layer; and
a mesh layer disposed along the absorbent layer on a side opposite the adhesive layer;
the mesh layer color-coded to indicate which of the plurality of compounds is integrated with the absorbent layer.

6. The bandage of claim 5, the compound comprising honey, the mesh layer color-coded amber.

7. The bandage of claim 5, the compound comprising tea tree oil, the mesh layer color-coded green.

8. The bandage of claim 5, the compound comprising colloidal oatmeal, the mesh layer color-coded tan.

9. The bandage of claim 5, the compound comprising baking soda, the mesh layer color-coded white.

10. The bandage of claim 5, the compound comprising a hemostatic agent, the mesh layer color-coded violet.

11. The bandage of claim 5, further comprising:
a film layer defining a first major face and a second major face, the first major face comprising one or more design elements and coupled to the backing layer;
the adhesive layer disposed along the second major face; and the absorbent layer disposed along the second major face at an interface.

12. The bandage of claim 11, the one or more design elements comprising a plurality of swirls.

13. A bandage, comprising:
a backing layer;
a film layer defining a first major face and a second major face, the first major face comprising one or more design elements and coupled to the backing layer;
an adhesive layer disposed along the second major face; and
an absorbent layer disposed along the second major face at an interface;
the interface to release the absorbent layer when the absorbent layer absorbs a wetting agent.

14. The bandage of claim 13, the wetting agent comprising wound exudate.

15. The bandage of claim 13, the one or more design elements defining a graphical pattern.

16. The bandage of claim 15, the graphical pattern comprising an identifier indicating the interface is to release the absorbent layer when the absorbent layer absorbs the wetting agent.

17. The bandage of claim 15, the graphical pattern comprising a plurality of swirls.

18. The bandage of claim 13, the adhesive layer comprising a hydrophobic adhesive.

19. The bandage of claim 18, the hydrophobic adhesive comprising a silicone-based adhesive.

20. The bandage of claim 13, the adhesive layer comprising a hydrophyllic adhesive.

21. The bandage of claim 13, the adhesive layer continuous across the second major face.

22. The bandage of claim 13, the adhesive layer discontinuous across the second major face.

23. The bandage of claim 22, the adhesive layer discontinuous under the absorbent layer.

24. The bandage of claim 23, further comprising a second adhesive layer disposed along the interface.

25. The bandage of claim 24, the adhesive layer comprising an acrylic-based adhesive, the second adhesive layer comprising a silicone-based adhesive.
